(19) United States
(12) Patent Application Publication
(10) Pub. No.: US 2010/0040686 A1
(43) Pub. Date: Feb. 18, 2010

(54) BIOMATERIAL

(76) Inventors: Osamu Masaki, Osaka (JP); Toshikazu Shiba, Nagano (JP); Ryuichi Morishita, Osaka (JP); Koichi Imura, Osaka (JP); Yusumasa Akagawa, Osaka (JP)

Correspondence Address: NIXON & VANDERHYE, PC, 901 NORTH GLEBE ROAD, 11TH FLOOR, ARLINGTON, VA, 22203, US

(21) Appl. No.: 12/447,158

(22) PCT Filed: Oct. 25, 2007
(86) PCT No.: PCT/JP2007/071276

§ 371 Date: Apr. 24, 2009

(30) Foreign Application Priority Data
Oct. 27, 2006 (JP) 2006-292169
Jul. 27, 2007 (JP) 2007-195813

(51) Int. Cl. A61K 9/14; A61K 38/18; A61K 33/42; A61P 19/00

(52) U.S. Cl. 424/484; 514/12; 424/605

(57) ABSTRACT

The present invention provides a biomaterial which releases slowly a biologically active substance acting only on bone cell regeneration, in order to compensate for bone or alveolar bone lost due to surgery, accident or the like. Specifically, the invention relates to a biomaterial containing an osteogenic factor adsorbed on a porous material selected from hydroxyapatite, calcium phosphate, β-TCP (tricalcium phosphate [β-Ca₃(PO₄)₂]), coral, calcium carbonate, titanium oxide, alumina, zirconia, silicon nitride, and ceramics. This osteogenic factor is preferably polyphosphoric acid or a pharmacologically acceptable salt thereof, or else bone morphogenetic protein (BMP).

### TECHNICAL FIELD

The present invention relates to a biomaterial that release slowly biologically active substances acting only on bone cell regeneration, in order to compensate for bone or alveolar bone lost due to surgery, accident or the like, and a manufacturing method thereof.

### BACKGROUND

Conventionally, various materials have been used and new components have been studied to compensate for parts of bone or alveolar bone lost due to surgery, accident or the like and to restore the body's original function. Although these materials may serve to fill in physical gaps, they are still foreign objects that do not adapt well to the body after implantation, often causing pain and discomfort for the patient.

In order to resolve this problem and achieve true regeneration, it is desirable that the implanted material be compatible with the body, as described for example in Quintessence Dental Implantology, 11(6) 723-730 2004 as follows. “Amid the recent focus on regenerative medicine, there has also been much research in the dental field into bone tissue regeneration using tissue cell engineering. The three things that must be considered when regenerating biological tissue are 1) a carrier, 2) cells and 3) biologically active substances” (page 729, left column, line 19); “BMP, TGF, PDGF and various other factors are said to be useful for bone tissue regeneration” (page 729, line 31).

Based on this kind of thinking, a “composition for delivery of TGF-β capable of delivering a sustained amount of TGF-β to a bone tissue application site and thereby accelerating osteogenesis and new bone tissue formation in a bone defect site” is disclosed for example in JP-A 7-2691, while a “bone-inducing preparation containing transforming growth factor (TGF) β and tricalcium phosphate” is disclosed in JP-A 8-505548 (Japanese Patent Publication No. 3347144).

The problem is, however, that because TGF-β has various functions in the regulation of cell growth and differentiation, apoptosis, cell migration, extracellular matrix production and degradation and the like, acting as a regulatory factor in body maintenance and repair, it has effects other than bone cell regeneration, and may cause or exacerbate cancer in particular. For this reason there has been demand for new biomaterials capable of releasing biological substances that act only on bone cell regeneration.

### DISCLOSURE OF THE INVENTION

It is an object of the present invention to provide a true biomaterial capable of compensating for and regenerating parts of bone or alveolar bone lost due to surgery or accident, and restoring the body's original function, along with a manufacturing method therefor.

It is also an object to achieve a safe and reliable regeneration and recovery in a long term by a sustained release of biologically active substances.

The present invention is (1) a biomaterial containing an osteogenic factor adsorbed on a porous material.

The present invention also encompasses the use of this biomaterial (1) to compensate for loss of bone or alveolar bone.

### DETAILED DESCRIPTION OF THE INVENTION

The present invention includes the following preferred embodiments.

(2) The biomaterial according to (1), wherein the osteogenic factor is polyphosphoric acid or a pharmacologically acceptable salt thereof, or bone morphogenetic protein (BMP).

(3) The biomaterial according to (2), wherein the degree of polymerization of the polyphosphoric acid or pharmacologically acceptable salt thereof is 15 to 2000.

(4) The biomaterial according to (2) or (3), wherein the pharmacologically acceptable salt of polyphosphoric acid is a sodium salt or potassium salt.

(5) The biomaterial according to any of (1) through (4), wherein the adsorbed amount of the polyphosphoric acid or pharmacologically acceptable salt thereof is 5 mass % or less of the mass of the biomaterial.

(6) The biomaterial according to any of (2) through (5), wherein the bone morphogenetic protein is BMP-1 or BMP-7 (OP-1).

(7) The biomaterial according to any of (1) through (6), wherein the porous material is one or more selected from hydroxyapatite, calcium phosphate, β-TCP (tricalcium phosphate [β-Ca₃(PO₄)₂]), coral, calcium carbonate, titanium oxide, alumina, zirconia, silicon nitride, and ceramics.

(8) The biomaterial according to any of (1) through (7), wherein a pharmacologically active component is also adsorbed.

(9) The biomaterial according to any of (1) through (8), wherein a pharmacologically active component is also adsorbed, and this pharmacologically active component is an anti-cancer drug, BRM or antibiotics or the like.

(10) The biomaterial according to any of (1) through (9), wherein a pharmacologically active component is also adsorbed, and this pharmacologically active component is one anti-cancer drug selected from cisplatin, doxorubicin hydrochloride, mitomycin C, bleomycin and rapamycin, one BRM selected from OK-432, BCG, IL-2 and IFN, and/or one antibiotic selected from penicillin, cephalosporin, streptomycin, tetracycline, vancomycin and gentamicin.

(11) The biomaterial according to any of (1) through (10), for substituting for bone or alveolar bone loss.

(12) A method for manufacturing the biomaterial according to any of (1) through (11), including a step of impregnating a porous material with an aqueous solution of 5 mass % or less of an osteogenic factor.

Advantages of the biomaterial of the present invention are that it is capable of compensating for and regenerating parts of bone or alveolar bone lost due to surgery or accident, and restoring the body's original function, while allowing sustained release of a biologically active substance to thereby ensure reliable regeneration and recovery long-term.

### BRIEF DESCRIPTION OF THE DRAWINGS

FIG. 1 is an electron microscope image showing the surface of hydroxyapatite having desirable properties.

FIG. 2 is a graph showing sustained release of 1% polyphosphoric acid treated for adsorption on hydroxyapatite (Example 1).

FIG. 3 is a graph showing sustained release of 5% polyphosphoric acid adsorbed on hydroxyapatite (Example 1).

FIG. 4 is a graph showing sustained release of 10% polyphosphoric acid adsorbed on hydroxyapatite (Example 1).

FIG. 5 is a graph showing sustained release of a protein adsorbed on hydroxyapatite (Example 2).

FIG. 6 is a graph showing sustained release of DNA adsorbed in hydroxyapatite (Example 3).

FIG. 7 is a photograph showing the external appearance of the sample used in Example 5.

FIG. 8 is a photograph for explaining the experimental method in Example 5.

FIG. 9 is a photograph for explaining the method of measuring new bone in term of a percentage of intrapore tissue area in a cortical bone defect site.

FIG. 10 is a scanning electron microscope (SEM) image of a sample surface.

FIG. 11 is a scanning electron microscope (SEM) image of a sample surface.

FIG. 12 is a scanning electron microscope (SEM) image of a sample surface.

FIG. 13 is an optical microscope image (left photo: magnification 40×) of a sample surface.

FIG. 14 is an optical microscope image (left photo: magnification 40×) of a sample surface.

FIG. 15 is an optical microscope image (left photo: magnification 40×) of a sample surface.

FIG. 16 is an optical microscope image (left photo: magnification 40×) of a sample surface.

FIG. 17 is an optical microscope image (left photo: magnification 40×) of a sample surface.

FIG. 18 is an optical microscope image (left photo: magnification 40×) of a sample surface.

FIG. 19 is an optical microscope image (left photo: magnification 40×) of a sample surface.

FIG. 20 is an optical microscope image (left photo: magnification 40×) of a sample surface.

FIG. 21 shows the results of tissue histomorphometrical measurement after a 2-week observation period.

FIG. 22 shows the results of tissue histomorphometrical measurement after a 3-week observation period.

FIG. 23 is a graph showing sustained release of 25% polyphosphoric acid adsorbed on hydroxyapatite (Example 1).

### (Porous Material)

The porous material of the present invention is not limited as long as it is biocompatible with bone or alveolar bone and is of a material having multiple small pores, but specific examples include hydroxyapatite, calcium phosphate, β-TCP (tricalcium phosphate [β-Ca₃(PO₄)₂]), coral, calcium carbonate, titanium oxide, alumina, zirconia, silicon nitride, ceramics and the like.

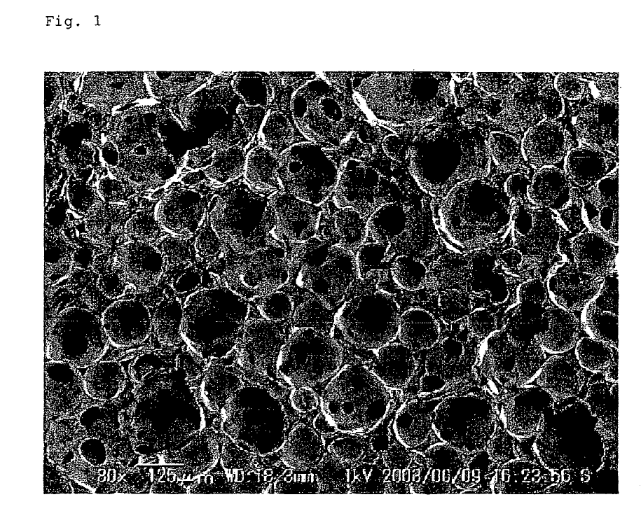

Of these, hydroxyapatite is preferred, and should preferably have the following physical properties for example. An electron microscope image of hydroxyapatite having the following physical properties is shown in FIG. 1.

Porosity: 72 to 78%

Pore diameter: 150 μm to 200 μm

Diameter of interconnection channel: 40 μm to 70 μm

Compressive strength: 12 MPa to 19 MPa

### (Osteogenic Factor)

The osteogenic factor of the present invention is not limited as long as it is a factor that promotes the proliferation, differentiation and migration of osteoblasts, and examples include polyphosphoric acid or pharmacologically acceptable salts thereof, or bone morphogenetic protein (BMP).

Polyphosphoric acid with a degree of polymerization of 15 to 2000 is preferred as the polyphosphoric acid or pharmacologically acceptable salt thereof. The pharmacologically acceptable salt is not particularly limited as long as it maintains its safety in the body, but examples include sodium and potassium salts.

The content of the polyphosphoric acid or pharmacologically acceptable salt thereof in the biomaterial is preferably 5 mass % or less or more preferably 3 mass % or less.

13 kinds of bone morphogenetic proteins (BMP) in the TGF-β superfamily are currently known as bone morphogenetic proteins. Among these, BMP-1 or BMP-7 (OP-1) is preferred. These bone morphogenetic proteins are available as reagents and the like, or may be manufactured in accordance with the methods described in the literature (Science 271, 360-362 and the like).

The content of the bone morphogenetic protein in the biomaterial is preferably 5 mass % or less or more preferably 1 mass % or less.

In the present invention, other pharmacologically active agents may also be adsorbed, and examples of such pharmacologically active agents include anti-cancer drugs, BRM, antibiotics and the like.

Examples of anti-cancer drugs include cisplatin, doxorubicin hydrochloride, mitomycin C, bleomycin, rapamycin and the like.

Examples of BRM include OK-432, BCG, IL2, IFN and the like.

Examples of antibiotics include penicillin, cephalosporin, streptomycin, tetracycline, vancomycin, gentamicin and the like.

### (Manufacturing Methods)

The method of manufacturing the biomaterial of the present invention has a step of impregnating a porous material with an aqueous solution of an osteogenic factor, wherein the concentration of the osteogenic factor in the aqueous solution is 5 mass % or less. In this impregnation step, deaeration may be performed in order to facilitate adsorption of the osteogenic factor by the porous material. A dehydration step or drying step may also be added after that as necessary.

### (Application and Method of Use)

The biomaterial of the present invention is used when compensating for and regenerating parts of bone or alveolar bone lost due to surgery, accident or the like, and may be used in a variety of ways and supplied in a variety of forms depending on the shape, size (area, volume) and the like of the defect site without limitations, but is normally supplied in the form of a block or granules. In the case of granules, the necessary amount can be packed directly into the defect site with pressure, or may be made into a slurry with distilled water, saline or the like and daubed onto the defect site. In the case of a block, it can be processed to fit the shape of the defect site and implanted.

### EXAMPLES

The present invention is explained in more detailed below using examples, but of course the present invention is not limited therein.

### Example 1

### Adsorption and Sustained Release of Sodium Polyphosphate on Hydroxyapatite

Blocks of hydroxyapatite (Neobone®, MMT Co., Ltd.) were immersed in 1% and 5% sodium polyphosphate aqueous solutions. The solutions were deaerated for 120 minutes with an aspirator (under conditions of −0.1 Mpa (Mega Pascal)) so that the aqueous solution would permeate the inside of the hydroxyapatite.

Once the sodium polyphosphate aqueous solution had completely permeated the hydroxyapatite, the hydroxyapatite blocks were removed from the aqueous solution and dehydrated by 2 minutes centrifugation at 3600 rpm to remove the aqueous solution remaining inside the hydroxyapatite. After this dehydrating step, the hydroxyapatite was dried for 3 days at 37° C. to obtain polyphosphoric acid adsorbed on hydroxyapatite as the biomaterial of the present invention.

1.8 μg of polyphosphoric acid per milligram was adsorbed by the hydroxyapatite that was adsorption-treated with the 1% sodium polyphosphate aqueous solution, while 10.8 μg of polyphosphoric acid per milligram was adsorbed by the hydroxyapatite that was adsorption-treated with the 5% sodium polyphosphate aqueous solution.

The content of polyphosphoric acid adsorbed on the hydroxyapatite was assayed as follows. 100 mg of the hydroxyapatite adsorbed with polyphosphoric acid was taken, thoroughly crushed and ultrasonicated for 1 hour in 0.1 ml of distilled water to elute the adsorbed polyphosphoric acid completely.

This was then centrifuged for 5 minutes at 10,000×g, and 20 μl of the supernatant was taken and subjected to acid hydrolysis by addition of 480 μl 2N-hydrochloric acid. 0.7 ml of a solution of ascorbic acid and ammonium molybdate at 1:6 was added to 0.3 ml of the hydrolyzed sample. The mixture was then maintained for 1 hour at 37° C. Absorbance was then measured at 820 nm to assay the phosphoric acid concentration, and the adsorbed polyphosphoric acid was calculated by the molar concentration per phosphoric acid residue.

Standard solutions of 0, 0.033, 0.067, 0.1, 0.133, 0.167 and 0.2 mM sodium hydrogenphosphate were used for the phosphoric acid assay, and a calibration curve was prepared from absorbance values of 0, 0.197, 0.371, 0.503, 0.610, 0.683 and 0.729, and used to determine the phosphoric acid concentration. The polyphosphoric acid concentration of each fraction after the elution test was also assayed after hydrolysis by the above mentioned method using molybdic acid.

The hydroxyapatite with adsorbed polyphosphoric acid was immersed in 0.1 ml of saline, and deaerated for 10 minutes so that the saline would permeate the inside of the hydroxyapatite. Once the saline had completely permeated the hydroxyapatite, it was set inside a glass column 1 cm in diameter and 2 cm long, and saline was passed through the column at a rate of 0.1 ml per minute using a medium-pressure liquid chromatograph (BioLogic Duo Flow, Biorad). The saline passed through of the column was fractioned in amounts of 0.25 ml with a fraction collector (Model 2110, Biorad) as the eluate.

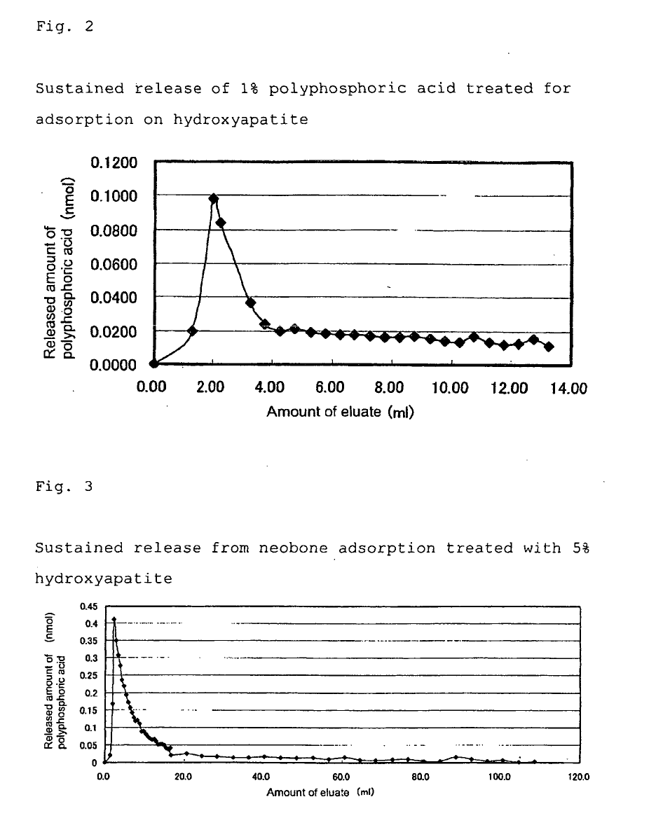

FIG. 2 shows a graph of changes in sustained release of polyphosphoric acid from hydroxyapatite that had been adsorption treated with 1% sodium polyphosphate aqueous solution. At 5 ml, the initial stage of elution from the column, excess adsorbed polyphosphoric acid (residue on the hydroxyapatite) was eluted, resulting in a temporary peak of high elution.

At this stage, the eluted amount per 1 μl of eluate was an average of about 0.06 nmol. By contrast, the eluted amount is roughly stabilized at not less than 5 ml, with concentration of 0.01 to 0.02 mmol of polyphosphoric acid eluted per 1 μl of eluate. The eluted amount declines gradually in accordance with the flow of eluate, but even when flow of the eluate exceeds about 13 ml, the eluted amount remains at 0.01 nmol or more.

This shows that, unlike the elution pattern for excess residual polyphosphoric acid (temporary peak of high elution), the polyphosphoric acid adsorbed by the hydroxyapatite is eluted at a fairly gradual rate.

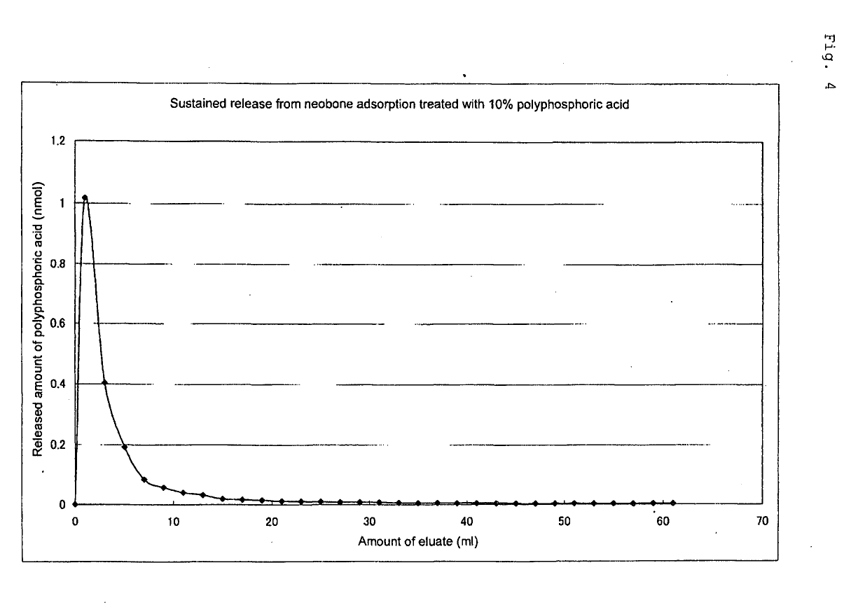

FIG. 3 shows a graph of changes in sustained release of polyphosphoric acid from hydroxyapatite that had been adsorption treated with 5% sodium polyphosphate aqueous solution. At 20 ml, the initial stage of elution from the column, excess adsorbed polyphosphoric acid (residue in the hydroxyapatite) was eluted, and a temporary peak of high elution was observed as in the case of adsorption treatment with 1% sodium polyphosphate aqueous solution.

The eluted amount of polyphosphoric acid per 1 μl of eluate changed greatly at this stage between 0.025 and 0.4 nmol. By contrast, the eluted amount of polyphosphoric acid was roughly stable between 20 and 60 ml of eluate, with concentration of 0.01 to 0.02 nmol eluted per 1 μl of eluate (saline). When the total flow of eluate was 20 ml or greater, the polyphosphoric acid was released at a rate similar to that obtained after adsorption treatment with 1% sodium polyphosphate aqueous solution, and it is thought that the adsorbed polyphosphoric acid was released stably during this 40 ml of flow. After that, the eluted amount declines bit by bit as the total amount of eluate increases, but even near 100 ml of flow 0.005 nmol or more was eluted.

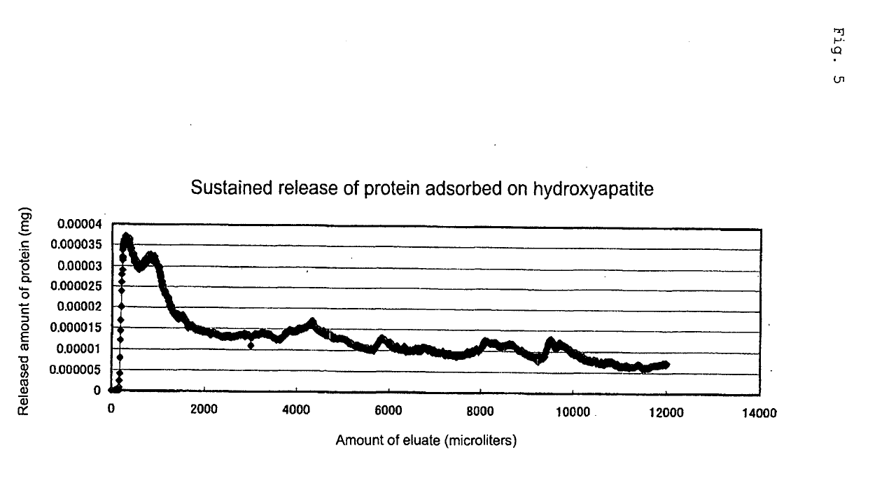

FIG. 4 shows a graph of changes in sustained release of polyphosphoric acid from hydroxyapatite that had been adsorption treated with 10% sodium polyphosphate aqueous solution. At 13 ml, the initial stage of elution, excess adsorbed polyphosphoric acid (residue in the hydroxyapatite) was eluted, and a peak of high elution was observed similar to those that occurred after adsorption treatment with 1% and 5% sodium polyphosphate aqueous solution.

The eluted amount of polyphosphoric acid per 1 μl of eluate changed greatly during this period between 0.03 and 1.0 nmol. By contrast, the eluted amount of polyphosphoric acid was roughly stable between 15 and 61 ml of eluate, with concentration of 0.003 to 0.019 nmol eluted per 1 μl of eluate (saline). When the total flow of eluate was 15 ml or greater, the polyphosphoric acid was released at a rate similar to that obtained after adsorption treatment with 1% and 5% sodium polyphosphate aqueous solution, and it is thought that the adsorbed polyphosphoric acid was released stably during this 46 ml of flow.

FIG. 23 shows a graph of changes in sustained release of polyphosphoric acid from hydroxyapatite that had been adsorption treated with 25% sodium polyphosphate aqueous solution. At 17 ml, the initial stage of elution, excess adsorbed polyphosphoric acid (residue in the hydroxyapatite) was eluted, and a peak of high elution was observed similar to those that occurred after adsorption treatment with 1%, 5% and 10% sodium polyphosphate aqueous solution.

The eluted amount of polyphosphoric acid per 1 μl of eluate changed greatly during this period between 4.03 μmol and 18.7 μmol. By contrast, the eluted amount of polyphosphoric acid was roughly stable between 19 and 41 ml of eluate, with concentration of 0.57 μmol to 0.97 μmol eluted per 1 μl of eluate (saline). When the total flow of eluate was 19 ml or greater, the polyphosphoric acid was released at a rate similar to that obtained after adsorption treatment with 1%, 5% or 10% sodium polyphosphate aqueous solution, and it is thought that the adsorbed polyphosphoric acid was released stably during this 22 ml of flow.

### Example 2

### Adsorption and Sustained Release of Protein (BSA) on Hydroxyapatite

Since BMP-1 or BMP-7, which can be used favorably in the present invention, is a protein, bovine serum albumin (BSA, Sigma) was used as a common protein to test adsorption of proteins onto hydroxyapatite and sustained release of the proteins from hydroxyapatite. A 328 mg of hydroxyapatite block was immersed in a 2 mg/ml BSA aqueous solution, and deaerated for 10 minutes with a vacuum pump so that the aqueous solution would permeate the inside of the hydroxyapatite.

Once the BSA solution had completely permeate the hydroxyapatite, the hydroxyapatite was removed from the aqueous solution, and centrifuged for 5 minutes at 8,000×g to remove residual aqueous solution from inside the hydroxyapatite. After this procedure, the hydroxyapatite was dried for 1 hour at 42° C. to obtain hydroxyapatite with adsorbed protein. 1.21 μg of BSA per milligram was adsorbed by the hydroxyapatite adsorption treated with BSA solution.

The adsorbed amount was calculated by subtracting the absorbance of the BSA solution remaining after adsorption from the absorbance at 280 nm of the BSA solution before adsorption treatment. The absorbance of the BSA solution was 0.555 at 2 mg/ml.

The hydroxyapatite with adsorbed BSA was immersed in 1 ml of saline, and deaerated for 10 minutes so that the saline would permeate the inside of the hydroxyapatite. After being completely permeated by the saline, the hydroxyapatite was set inside a glass column 1 cm in diameter and 2 cm long, and saline was passed through the column at a rate of 0.2 ml per minute using a medium-pressure liquid chromatograph (BioLogic Duo Flow, Biorad). The absorbance of the saline passing through of the column was measured continuously (every second) at 280 nm with a UV detector, and the eluted amount of BSA was assayed.

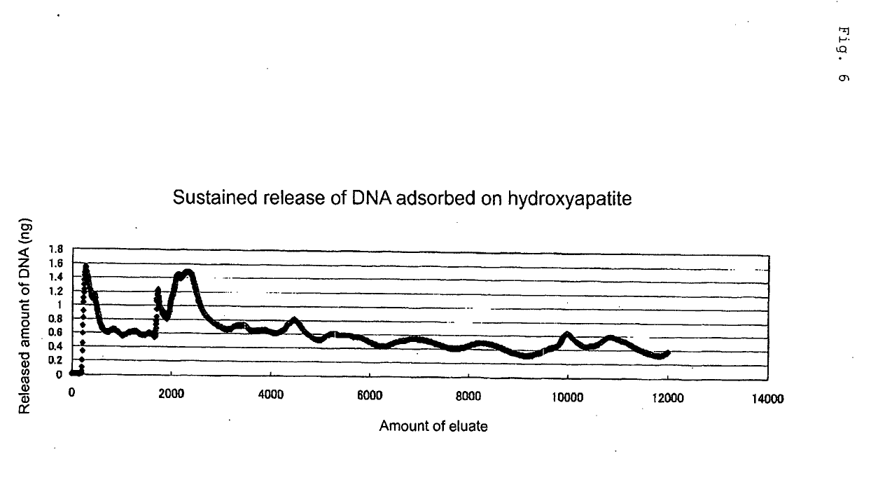

FIG. 5 shows a graph of changes in sustained release of BSA from hydroxyapatite that had been subjected to adsorption treatment using BSA. At 2.5 ml, the initial stage of elution from the column, excess adsorbed BSA (residue on the hydroxyapatite) was eluted, and the eluted amount rose to 36 ng in the same way as the polyphosphoric acid, resulting in a temporary peak of high elution.

By contrast, the eluted amount roughly stabilized at 2.5 ml of eluate and more, with BSA being eluted in the narrow range of 6 to 13 ng per 3.333 μl of eluate (saline). The eluted amount declined gradually in accordance with the flow of eluate, but the same amount was maintained up to 12 ml of flow.

This shows that BSA adsorbed on hydroxyapatite is released at a relatively stable rate after the initial flow of excess residual BSA. BSA is a typical protein with properties of common proteins. Since BMP-1 or BMP-7 is also a protein, adsorption and release of BMP-1 or BMP-7 from hydroxyapatite are made evident with results for adsorption and release of BSA.

### Example 3

### Adsorption and Sustained Release of nucleic Acids (DNA) from Hydroxyapatite

To test adsorption and sustained release of nucleic acids from hydroxyapatite, salmon testis DNA (deoxyribonucleic acid sodium from salmon testis (filamentous), for biochemical use, Wako Pure Chemical) was ultrasonicated and broken down into lengths of about 100 to 200 nucleotide residues on average for purposes of use.

A 150 mg of block hydroxyapatite was submerged in a 1 mg/ml DNA solution, and deaerated for 10 minutes with a vacuum pump so that the aqueous solution would permeate into the inside of the hydroxyapatite. Once the DNA solution had permeated completely, the hydroxyapatite was removed from the aqueous solution and centrifuged for 5 minutes at 8,000×g to remove aqueous solution remaining in the hydroxyapatite. Following this operation, the hydroxyapatite was dried for 1 hour at 42° C. to obtain hydroxyapatite with adsorbed DNA. 0.2 μg of DNA per milligram was adsorbed by the hydroxyapatite that had been adsorption treated with the DNA solution.

The adsorbed amount was calculated by subtracting the absorbance of the DNA solution remaining after adsorption from the absorbance at 254 nm of the DNA solution before adsorption treatment. The absorbance of the DNA solution was 20 in a 1 mg/ml solution.

The hydroxyapatite with adsorbed DNA was immersed in 1 ml of saline, and deaerated for 10 minutes so that the saline would permeate the inside of the hydroxyapatite. After the saline had completely permeated the hydroxyapatite, it was set in a glass column 1 cm in diameter and 2 cm long, and saline was passed through the column at a rate of 0.2 ml a minute using a medium-pressure liquid chromatograph (BioLogic, Biorad). The absorbance of the saline passed through the column was measured continuously (every second) at 254 nm with a UV detector, and the eluted amount of DNA was assayed. The DNA concentration was given as 1 absorbance unit=50 μg/ml.

FIG. 6 shows a graph of changes in sustained release of DNA from hydroxyapatite that had been adsorption treated with DNA solution. At the initial stage of elution from the column, 3.5 ml, excess adsorbed DNA (residue on the hydroxyapatite) was eluted, and the eluted amount reached an unstable, extremely high peak. The maximum eluted amount at this stage was about 1.6 ng.

By contrast, the eluted amount roughly stabilized at 3.5 ml of eluate and more, and fluctuated between 0.3 and 0.8 ng up to 12 ml of eluate. The eluted amount then declined gradually in accordance with the flow of eluate, but was still 0.4 ng even after 11 ml of eluate. This shows that once the excess residual DNA was eluted (observed as an elution peak), the DNA adsorbed by the hydroxyapatite was gradually released at a steady rate.

### Example 5

### Materials and Methods

### 1. Materials

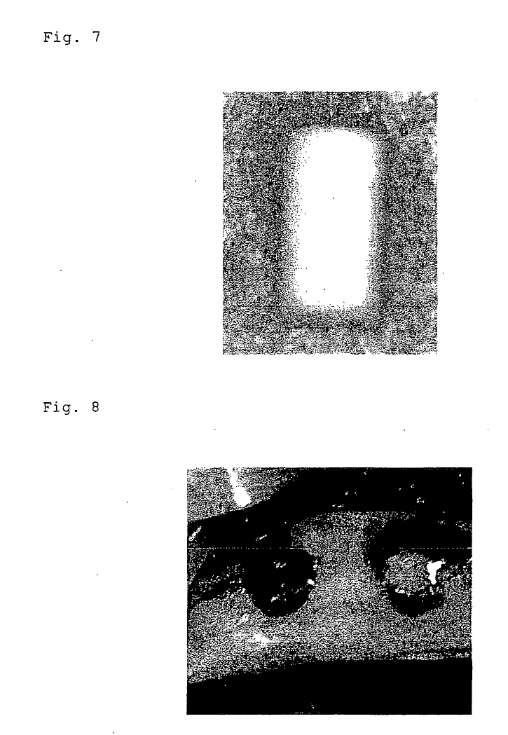

8 New Zealand white rabbits (2 to 2.5 kg) were used as the experimental animals. A drug-eluting artificial bone (hydroxyapatite with adsorbed polyphosphoric acid, polyphosphoric acid concentration 1, 5, 25%, hereunder abbreviated as P-IPHA) and interconnected porous hydroxyapatite (Neobone™, Covalent Materials, hereunder abbreviated as IPHA) were used as the materials, which were molded into cylinders (3 mm in diameter and 5 mm in height) and used as the samples (FIG. 7).

### 2. Observation of Surface Structure

In order to confirm the surface properties of the 5% P-IPHA and IPHA, the surfaces were subjected to Pt—Pd spattering and observed at an angle of 45° to the sample surface with a scanning electron microscope (JSM-6300, JEOL Datum, hereunder abbreviated as SEM).

### 3. Evaluation of Bone Formation Ability

The animal's left femur was exposed, the cortical bone was punctured with a round bar, and two bone pits were formed to a depth of 5 mm with a drill bar (3 mm in diameter) to create the specified bone defects. P-IPHA and IPHA were each implanted in one of these bone pits (FIG. 8).

After implantation, the fascia were sutured with polylactide absorbable thread, and the skin flaps with silk thread to close the wounds. All these surgical procedures were performed under general anesthesia using 1.0 ml/kg of 1.0 mg/ml medetomidine hydrochloride (Domitor®, Meiji Seika) by intramuscular injection and 0.5 ml/kg of 50 mg/ml pentobarbital sodium (Nembutal®, Dainippon Sumitomo) by intravenous injection combined with local anesthesia using 2% Lidocaine containing epinephrine (Xylocalne®, Fujisawa). To prevent infection, 0.5 ml/day of an enrofloxacin preparation (Baytril®, Bayer Japan) was administered intramuscularly for 1 week after surgery. One week after implantation, the same procedure was performed on the right side. Two weeks after that (3 weeks after the left-side procedure), the animals were given 2500 units of pentobarbital sodium and a blood coagulation inhibitor (Novo-Heparin Injection 1000®, Hoechst Marion Roussel Japan), the chests were opened, the pericardium was detached, and the animals were perfusion fixed by injection of biological saline and 10% neutral formalin through the aorta via the ventricles. Both femurs were then removed, and immersed for 48 hours in fixing solution.

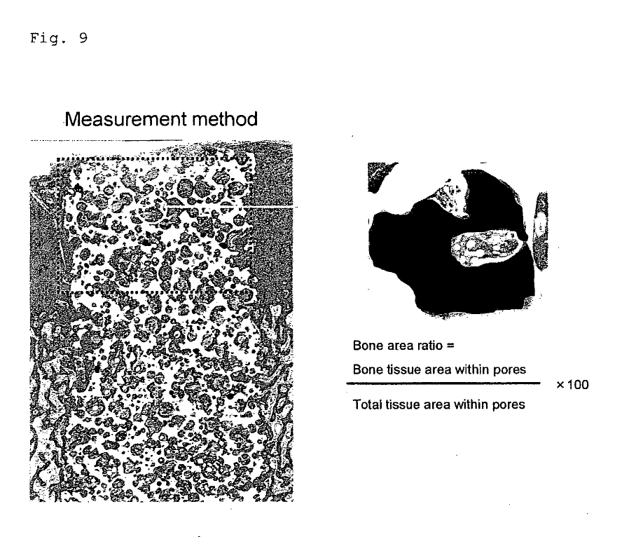

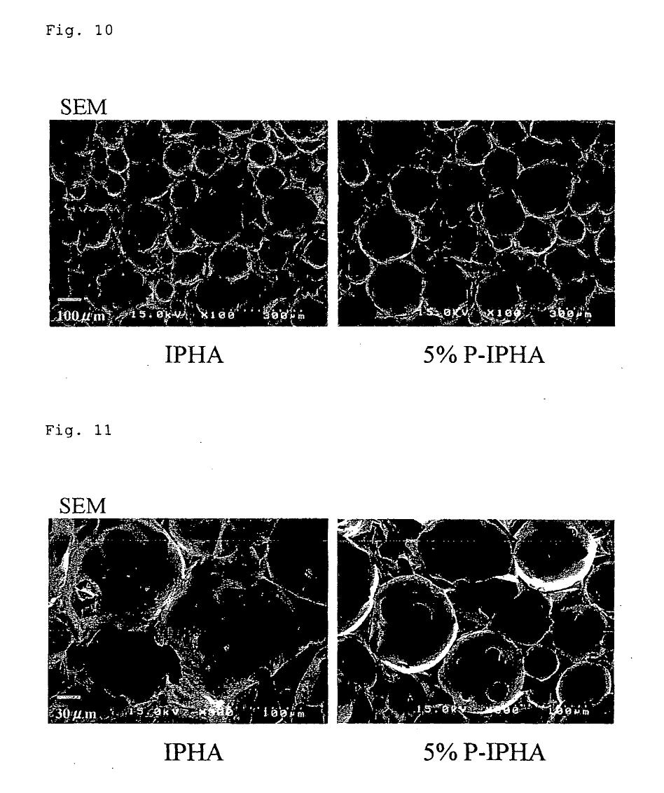

The extracted femurs were cut and trimmed with a hard tissue microcutting machine (cutting machine for hard tissue BS-3000, EXAKT APARATEBAU), to obtain tissue blocks of each bone pit including the samples. These were decalcified by being immersed for 3 days in a rapid decalcifying solution (KC-X®, Shionogi), then dehydrated with alcohol and permeated with xylene, and finally embedded in paraffin. Next, tissue slices about 5 μm thick were prepared with a microtome, stained with hematoxylin-eosin (HE stain), and observed under an optical microscope. To measure the tissue histomorphometrically, the HE-stained specimens were digitalized and entered into a personal computer, and new bone as a percentage of the tissue area in the pores of the cortical defect site was calculated as the bone area ratio (FIG. 9) using image analysis software (Image J, National Institutes of Health). The bone area ratio value was analyzed statistically with a significance level of 5% by one-way analysis of variance and Tukey HSD multiple comparison test (n=4).

### (Results)

### I. Observation by Scanning Electron Microscopy (SEM)

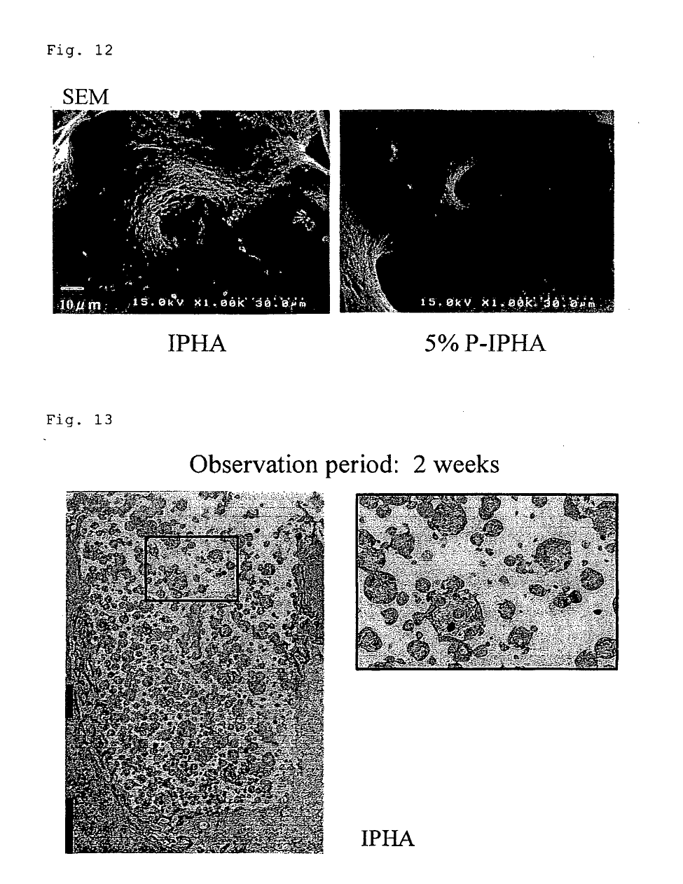

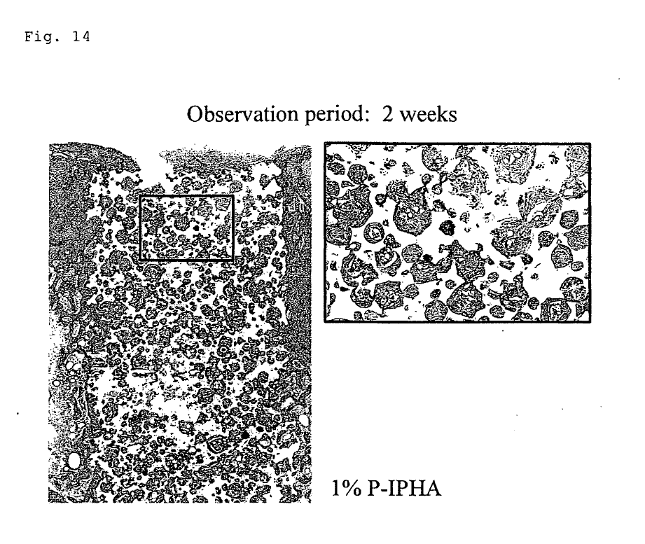

In an observation of the sample surface structures by SEM, the structures of the pores and interconnected pores were similar in the P-IPHA and IPHA (FIGS. 10, 11). In terms of surface properties, the P-IPHA was somewhat smoother than the IPHA (FIG. 12).

### II. Histological Observation

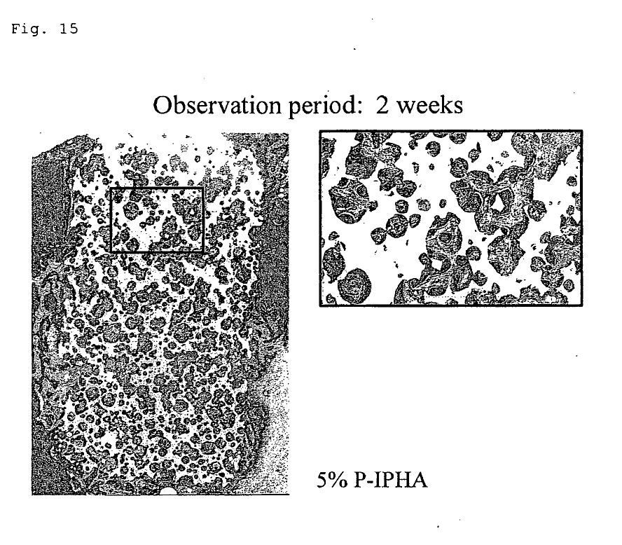

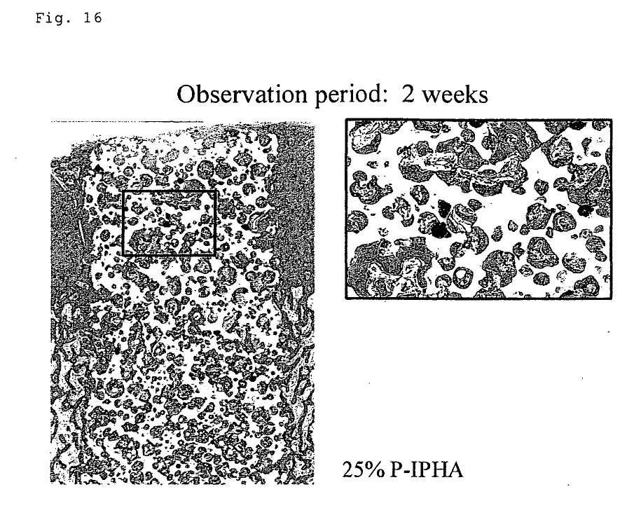

Under an optical microscope, the insides of the pores of the samples were filled with new bone and fibrous tissue in all groups 2 weeks after implantation, and layer of cubic osteoblast-like cells were also seen on the newly-generated bone surfaces. Moreover, the bone tissue formed inside the pores was in direct contact with the pore surfaces. In the 5% and 25% P-IPHA groups in particular, there was obvious new bone formation in the central part of the cortical bone defects (FIGS. 13, 14, 15, 16).

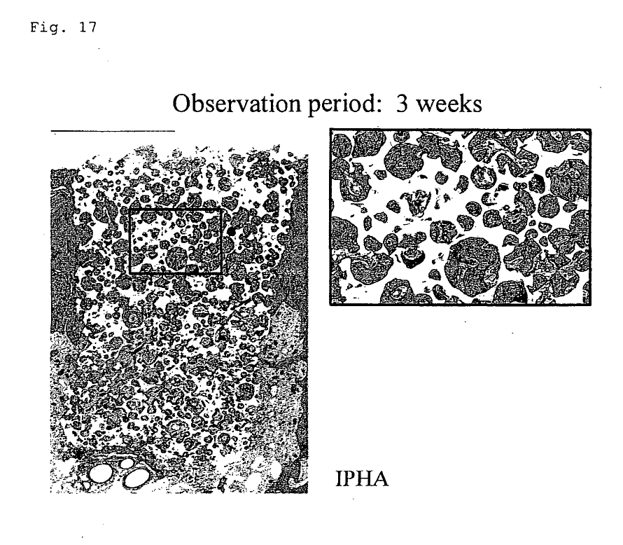

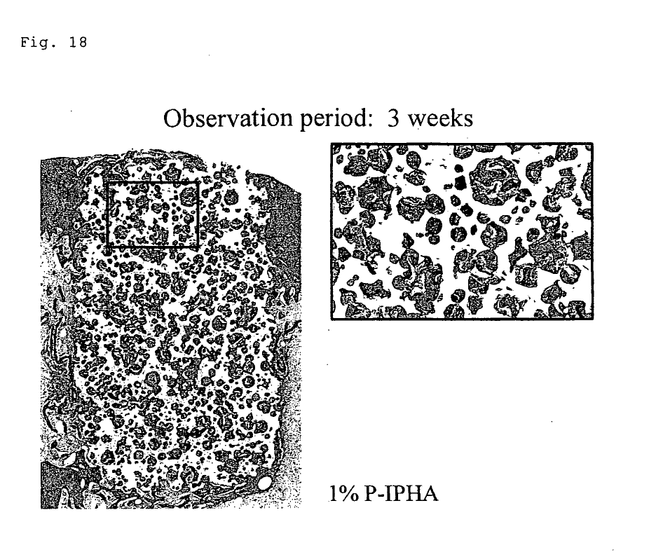

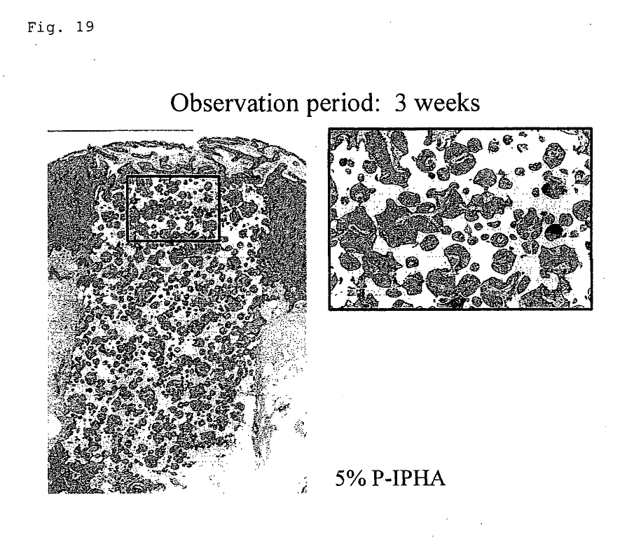

3 weeks after implantation, the pores inside the samples were occupied by large quantities of bone tissue, and mature osteoblast-like cells and the like were observed (FIGS. 17, 18, 19, 20).

### III. Evaluation of Osteogenic Ability

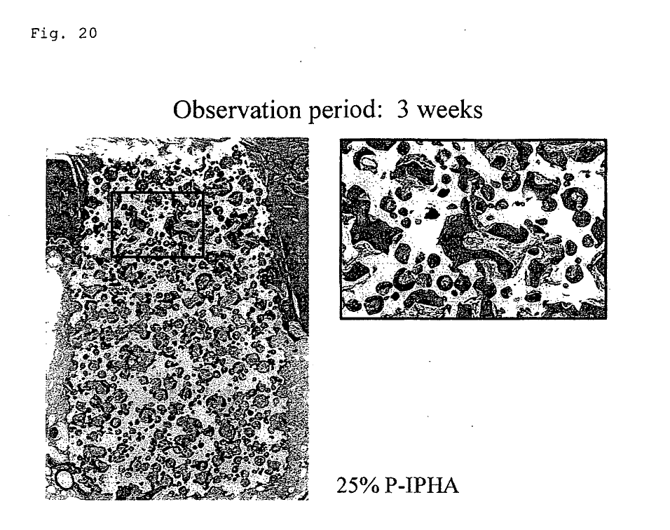

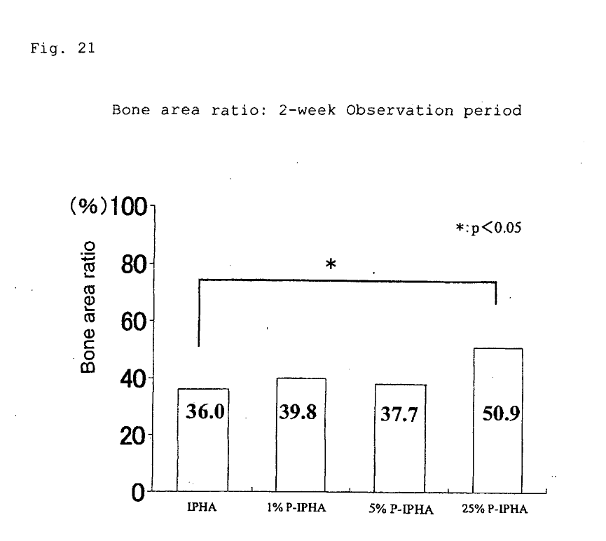

The results of tissue-histomorphometrical measurement are shown in FIGS. 20 and 21. The bone area ratio 2 weeks after implantation was 36.0%, 39.8%, 37.7% and 50.9% in the IPHA and 1%, 5% and 25% P-IPHA groups, and the value of the 25% P-IPHA group was significantly greater than that of the IPHA group (p<0.05) (FIG. 21). The 5% P-IPHA group was excluded from statistical treatment because of an insufficient n value due to bone fracture during the 2-week observation period.

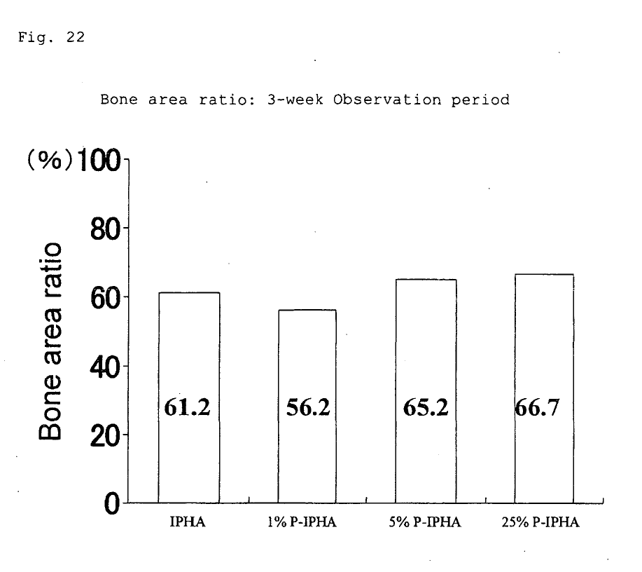

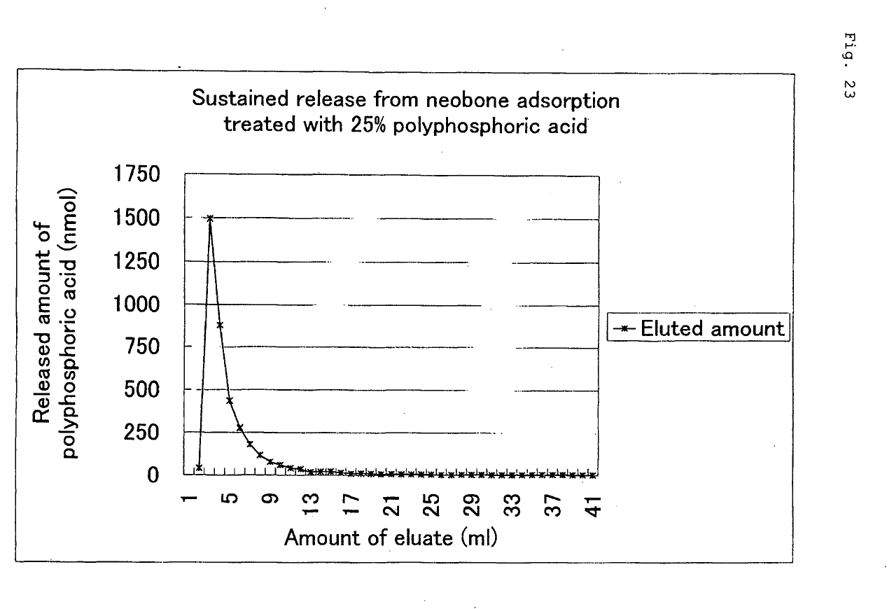

The bone area ratios of 3 weeks after the implantation were 61.2%, 56.2%, 65.2% and 66.7% in IPHA, 1%, 5% and 25% P-IPHA groups, respectively. No statistically significant differences were observed between all groups (FIG. 22).

### (Discussion)

Since blockage and the like of interconnected pores due to adsorption of the polyphosphoric acid on the sample surfaces did not occur, it is thought that P-IPHA has a interconnected porous structure like that of IPHA, and therefore has a similar bone-conducting ability.

After two weeks of observation, 25% P-IPHA showed significantly greater bone formation than IPHA. The period up to two weeks after implantation is the initial stage of bone formation when granulation tissue forms, followed by the beginning of calcification. Phosphoric acid is known to have the ability to induce bone by causing differentiation of undifferentiated stem cells into osteoblasts. Therefore, it is thought that when a sample is implanted, the local increase in phosphoric acid concentration induces undifferentiated stem cells contained in bone marrow cells that aggregate during the tissue recovery period to differentiate into osteoblasts, thereby promoting bone formation. No significant difference in bone formation in all groups after 3 weeks was observed. It is thought that in a closed bone defect such as the current experimental model, the sample creates a definite space that allows for almost complete bone formation and maturation due to bone conduction from the surrounding bone.

Thus, the newly-developed drug-eluting artificial bone (P-IPHA) has a bone formation-promoting effect due to the adsorbed polyphosphoric acid, and since there is no structural change in the communicating pores due to the adsorbed polyphosphoric acid, it is thought to be an excellent bone implantation material with superior bone-conducting and bone-inducing ability.

1. A biomaterial comprising an osteogenic factor adsorbed on a porous material.

2. The biomaterial according to claim 1, wherein said osteogenic factor is polyphosphoric acid or a pharmacologically acceptable salt thereof, or bone morphogenetic protein (BMP).

3. The biomaterial according to claim 2, wherein the degree of polymerization of said polyphosphoric acid or pharmacologically acceptable salt thereof is 15 to 2000.

4. The biomaterial according to claim 2, wherein said pharmacologically acceptable salt of polyphosphoric acid is a sodium salt or potassium salt.

5. The biomaterial according to claim 1, wherein the adsorbed amount of said polyphosphoric acid or pharmacologically acceptable salt thereof is 5 mass % or less of the mass of the biomaterial.

6. The biomaterial according to claim 2, wherein said bone morphogenetic protein is BMP-1 or BMP-7 (OP-1).

7. The biomaterial according to claim 1, wherein said porous material is one or more selected from the group consisting of hydroxyapatite, calcium phosphate, β-TCP (tricalcium phosphate [β-Ca₃(PO₄)₂]), coral, calcium carbonate, titanium oxide, alumina, zirconia, silicon nitride, and ceramics.

8. The biomaterial according to claim 1, wherein a pharmacologically active component is also absorbed.

9. The biomaterial according to claim 1, wherein a pharmacologically active component is also adsorbed, and said pharmacologically active component is an anti-cancer drug, BRM or antibiotic.

10. The biomaterial according to claim 1, wherein a pharmacologically active component is also adsorbed, and said pharmacologically active component is one anti-cancer drug selected from the group consisting of cisplatin, doxorubicin hydrochloride, mitomycin C, bleomycin and rapamycin, one BRM selected from the group consisting of OK-432, BCG, IL-2 and IFN, or one antibiotic selected from the group consisting of penicillin, cephalosporin, streptomycin, tetracycline, vancomycin and gentamicin.

11. The biomaterial according to claim 1, for compensating for bone or alveolar bone loss.

12. A method for manufacturing the biomaterial according to claim 1, comprising a step of impregnating a porous material with an aqueous solution of 5 mass % or less of an osteogenic factor.
